(19) United States
(12) Patent Application Publication
(10) Pub. No.: US 2009/0076381 A1
(43) Pub. Date: Mar. 19, 2009

(54) METHOD OF DISCRIMINATING RIGHT OR LEFT FOR BREAST REGIONS

(76) Inventors: Wataru Motoki, Tokyo (JP); Mamoru Umeki, Tokyo (JP); Eiichi Akagi, Tokyo (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/915,136

(22) PCT Filed: Apr. 11, 2006
(86) PCT No.: PCT/JP2006/307608

§ 371 Date: Nov. 20, 2007

(30) Foreign Application Priority Data
May 25, 2005 (JP) 2005-152204
May 25, 2005 (JP) 2005-152209

(51) Int. Cl. A61B 5/05; G06K 9/00

(52) U.S. Cl. 600/425; 382/128

(57) ABSTRACT

A method of discriminating left or right for breast regions, including generating data of plural breast images when a left breast region and a right breast region are alternately imaged in an order and are repeatedly imaged in various imaging directions in the same order by image generating sections arranged in a certain direction. A breast image is specified which is capable of being discriminated whether the imaged breast region is left or right among the plural breast images, and it is discriminated whether the left breast region or the right breast region is imaged concerning other breast images, based on the specified breast image and the order of imaging of the plural breast images of a single patient.

### TECHNICAL FIELD

The present invention relates to a method of discriminating whether an imaged portion in each breast image is a right breast region or a left breast region.

### BACKGROUND OF THE INVENTION

There has been developed an imaging system wherein an X-ray imaging apparatus for imaging breast images and its control apparatus are connected through a communication network, and operations for controlling imaging are made easy (for example, see Patent Document 1).

In this imaging system, there is used a CR cassette having a built-in phosphor plate on which a stimulable phosphor layer is formed, and the phosphor plate is scanned with a laser beam after causing the phosphor plate to absorb X-rays transmitted through a breast, so that radiation energy accumulated in the phosphor layer may be emitted as fluorescence, whereby, breast image data can be obtained by converting the emitted fluorescence on a photoelectric basis.

In recent years, an FPD (Flat Panel Detector) is widely used as a recording device for breast images, in place of a CR cassette. This FPD is one that has a plurality of X-ray detecting elements arranged on a two-dimensional basis, and accumulates X-rays transmitted through a breast on the X-ray detecting elements to generate breast image data from electric signals corresponding to an amount of accumulated X-rays, directly. Therefore, a technician does not need to move to a place of installation for a reading apparatus in a CR system, because breast image data are generated directly, resulting in more smooth overall imaging operations and in less load for a technician in the course of imaging, compared with an occasion where the CR system.

On the other hand, information of instructions concerning imaging that is called imaging order information is issued by the request of a doctor prior to imaging, and imaging operations are advanced by a technician based on this imaging order information, in the aforesaid imaging system. The imaging order information represents information of instructions including information concerning a patient such as a name and the sexuality of a patient to be imaged, information concerning imaging such as an imaging region, an imaging direction and a method of imaging, and information of inspections such as a date of inspection and a name of the doctor in charge.

After breast images are generated by imaging conducted based on the imaging order information, there is conducted processing in the imaging system to cause the imaging order information to be accompanied by the breast images as information concerning breast images. After that the breast images are caused to be data base and are controlled based on the accompanying information.

Patent Document 1: Japanese Patent Publication Open to Public Inspection No. 2002-158862

### DISCLOSURE OF THE INVENTION

### Problems to be Solved by the Invention

When imaging breast images, imaging operations are conducted in plural imaging directions for right and left breasts for a single patient, in many cases. Therefore, a plurality of CR cassettes are sometimes used for continuous imaging operations. When a plurality of CR cassettes are used, if the relationship between the CR cassette and the imaging by the imaging order information is unclear, it becomes impossible to recognize which imaging order information should be given to the breast image that has been read out of the CR cassette, in the imaging system. Therefore, it becomes necessary to provide the processing of the setting registration (which is called cassette registration) that causes imaging order information and a natural ID of the CR cassette to correspond to each other before imaging or after imaging.

However, when the CR cassette registration is conducted before imaging, a technician needs to conduct imaging by using the cassette which has been registered for the imaging order information, which makes the relationship of correspondence between the imaging order information to be used for imaging and the registered cassette to be stored. On the other hand, when the CR cassette registration is conducted after imaging, it is still necessary to store the CR cassette and the imaging where the CR cassette was used in the course of imaging, which is complicated. Further, there is a possibility of registration failure caused by wrong storage.

In the technology of the foresaid Patent Document 1, image processing corresponding to imaging region (right and left of breast) and imaging direction is conducted based on imaging order information accompanying to breast images. However, when the aforesaid registration failure takes place for the CR cassette, it is considered that appropriate image processing will not be conducted.

Further, when conducting imaging by using FPD in place of a CD cassette, if the relationship between the generated breast images and the imaging order information becomes unclear, it becomes impossible to recognize the relationship between the medical images generated by FPD and the imaging order information through which the medical images were obtained. Therefore, it is necessary to conduct setting registration for the imaging order information corresponding to medical images generated by FPD before imaging or after imaging.

However, it is troublesome to leave the imaging room and to select on a control apparatus each time the imaging direction and the imaging region are changed, resulting in a load of operations for a technician.

The present invention has been achieved in view of the aforesaid problems, and its objectives are to discriminate easily whether an imaged portion in each of plural breast images is a left breast or a right breast and to dispense with registration work for imaging order information by a technician.

### Means to Solve the Problems

The aforesaid objectives of the invention are achieved by the following structures.

### (1) Structure 1

A method of discriminating right or left of breast region including a generating process that generates data of breast images in the order of imaging conducted alternately for left and right for both of left and right breasts of a patient, and a discriminating process wherein a breast image capable of being discriminated whether an imaged breast region is a left breast or a right breast among the aforesaid generated plural breast images is specified, and other breast images are discriminated whether each of them is left or right breast region, based on this specified image and on the imaging order for the aforesaid plural breast images.

### (2) Structure 2

The method of discriminating right or left of breast regions according to Structure 1, wherein the breast image capable of being discriminated whether an imaged breast region is left or right is a breast image obtained through imaging in the oblique direction.

### (3) Structure 3

The method of discriminating right or left of breast regions according to Structure 2, wherein the aforesaid plural breast images are breast images obtained through imaging in the oblige direction and vertical direction for each of left and right breast regions.

### (4) Structure 4

The method of discriminating right or left of breast regions according to Structure 2 or 3, wherein the imaging direction in each of other breast images is discriminated based on the breast image obtained through imaging in the foresaid oblique direction, in the aforesaid discriminating process.

### (5) Structure 5

The method of discriminating right or left of breast regions according to Structure 4, wherein, when each breast image is arranged in the imaging order based on breast region of breast image taken through imaging in the oblique direction, a breast region in each of other breast images is discriminated so that a breast region of each breast image may be alternately for left and right, in the aforesaid discriminating process.

### (6) Structure 6

The method of discriminating right or left of breast regions according to Structure 5, wherein when a user inputs information of left or right for a breast region in a breast image about the breast image taken through imaging in the aforesaid oblique direction, a breast region of each of other breast images is discriminated to be a left breast or a right breast based on left and right information of the inputted breast region, in the aforesaid discriminating process.

### (7) Structure 7

The method of discriminating right or left of breast regions according to Structure 5, wherein an image is analyzed for each of the breast images, an breast image taken through imaging in the oblique direction is discriminated based on the results of the image analysis, and a breast region of other breast image is discriminated whether it is left or right based on the discriminated breast image in the oblique direction.

### EFFECTS OF THE INVENTION

In the Structures (1)-(5), left or right of a breast region in a breast image and an imaging direction can easily be discriminated, even when breast images taken through imaging include breast images which are difficult to discriminate in terms of left and right of a breast region, like breast images taken through imaging in a CC direction. Owing to this, a load of imaging operations for a technician can be reduced substantially, because it is possible to dispense with registration work for imaging order information.

The Structure (6) makes it possible for a technician to specify a breast image in the oblique direction through visual observation.

The Structure (7) makes it possible to discriminate automatically left or right of a breast region in a breast image from image analyses, resulting in excellent work efficiency of a technician.

### BRIEF DESCRIPTION OF THE DRAWINGS

FIG. 1 is a diagram showing breast images taken through imaging carried out alternately for a left breast and a right breast.

FIG. 2 is a diagram showing the system structure of a breast imaging system in the First Embodiment.

FIG. 3 is a diagram showing an imaging apparatus in FIG. 2.

FIG. 4 is a side view of the imaging apparatus.

FIG. 5 is a side view of the imaging apparatus in the case of applying a phase contrast method in the First Embodiment.

FIG. 6 (a) is a diagram showing FPD example having a handle portion, and

FIG. 6 (b) is a diagram showing FPD example having a switch button.

FIG. 7 is a diagram showing a schematic structure of FPD.

FIG. 8 is a diagram showing an internal structure of a control apparatus.

FIG. 9 is a flowchart illustrating a flow of processing in a breast imaging system of the First Embodiment.

FIG. 10 is a diagram showing an example of a viewing surface for the results of imaging displayed on a control apparatus.

FIG. 11 is a diagram showing examples of imaging patterns.

FIG. 12 is a diagram showing an example of a viewing surface for the results of judgment displayed on a control apparatus.

FIG. 13 is a diagram showing the other system structure in the First Embodiment.

FIG. 14 is a diagram showing the system structure in a breast imaging system of the Second Embodiment.

FIG. 15 is a side view of the imaging apparatus in the case of applying a phase contrast method in the Second Embodiment.

FIG. 16 is a diagram illustrating an example of loading a cassette on an imaging stand.

FIG. 17 is a flowchart illustrating a flow of processing in a breast imaging system of the Second Embodiment.

FIG. 18 is a diagram showing the other system structure in the Second Embodiment.

### EXPLANATION OF SYMBOLS

100 Breast imaging system
10 Imaging apparatus
30. Control apparatus
31. Control section
32. Operation section
33. Display section
36. Image memory
50. FPD
70. Reading apparatus

### PREFERRED EMBODIMENT OF THE INVENTION

### First Embodiment

For breast images, imaging is carried out in one direction such as an oblique direction {hereinafter abbreviated as an MLO direction) in a simple examination for each of a left breast and a right breast. However, in case of an ordinary medical examination, imaging is carried out frequently for each of a left breast and a right breast in different directions such as the MLO and vertical directions (hereinafter abbreviated as CC). In case of imaging, a breast is pressed, and this pressure sometimes causes a pain. When conducting imaging continuously several times, therefore, it is general that imaging is conducted alternately for left and right such as, for example, the order of left→right→left→right.

For example, if imaging is conducted for the total four times in MLO and CC directions for each of a left breast and a right breast,

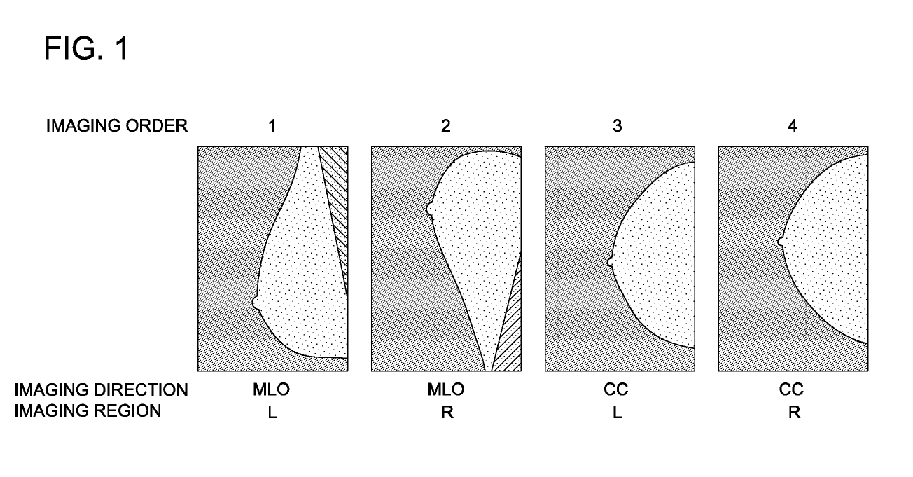

four breast images generated from the aforesaid imaging show the pattern in FIG. 1 when they are arranged in the order of imaging. Incidentally, in this case, it is assumed that imaging was conducted by changing a breast region (breast) in the order of left→right. Further, arrangement of FPD with respect to the patient and reading conditions of the data stored in FPD are made to be the same, and conditions for generating each breast image are unified. In FIG. 1, each breast image which is generated under the unified generating conditions is arranged so that one side where a patient is brought into contact with FPD may be positioned at the right side in each format.

As shown in FIG. 1, for the breast image concerning MLO, it is possible to judge that the imaging direction is MLO based on a form of the breast or on presence of a pectoral muscle, and its imaging region can be discriminated whether is a left breast or a right breast based on the fact that the breast is arranged upside down, through visual observation of a technician or through pattern recognition processing by a computer.

On the other hand, for the breast image concerning CC, it is difficult to discriminate through visual observation or a pattern recognition, because a breast shape is similar and the position of arrangement is the same in the breast image for both the left and right breasts. However, under the assumption that imaging is conducted alternately for the left breast and the right breast, as stated above, it is possible to discriminate that an imaged region of the breast image in the imaging order 3 is a left breast, from the imaging order and the imaged region of the breast image of MLO and an imaged region of the breast image in the imaging order 4 is a right breast. Further, the imaging direction of other breast images excluding MLO is CC.

Namely, under the assumption that imaging is conducted alternately for left and right and the arrangement of FPD with respect to a patient is made to be the same, if the breast image of MLO capable of being discriminated whether an imaged region is left or right among plural breast images becomes clear, it is possible to discriminate the imaged region and the imaging direction for other breast images.

Based on the foregoing, there will be explained an example to discriminate an imaged region and an imaging direction for other breast images from breast images of MLO, after conducting data generation for breast images under the same conditions, in the present embodiment.

First, the constitution will be explained.

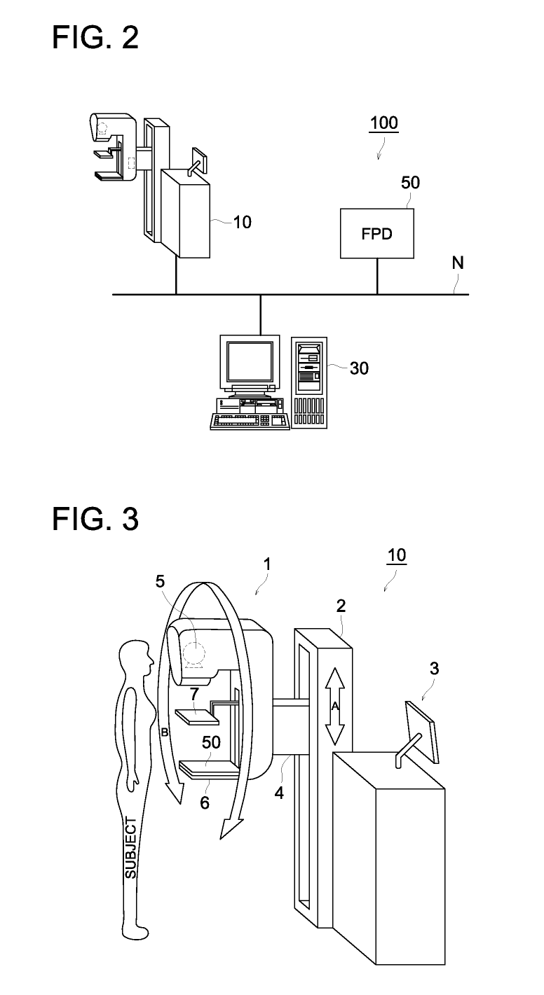

FIG. 2 shows the system structure of breast imaging system 100.

The breast imaging system 100 is one that conducts imaging a breast by using FPD, and generates its breast image data. As shown in FIG. 2, the breast imaging system 100 is composed of imaging apparatus 10, control apparatus 30 and FPD 50, and these respective apparatuses 10, 30 and 50 are connected each other through communication network N. Incidentally, FPD 50 may also be of the structure capable of connecting directly to the control apparatus 30.

The imaging apparatus 10 is one to irradiate X-ray on a breast of a patient, for conducting imaging.

FIG. 3 shows imaging apparatus 10.

As shown in FIG. 3, the imaging apparatus 10 is constructed so that imaging section 1 that conducts imaging is formed to be in a form of an arm, and the imaging section 1 is constructed to be capable of rising and falling along columnar support 2 and to be capable of rotating through supporting shaft 4. It further has main body portion 3 for rising, falling and rotating of the imaging section 1 and for communication operations with control apparatus 30, and a technician can conduct imaging operations through this main body portion 3.

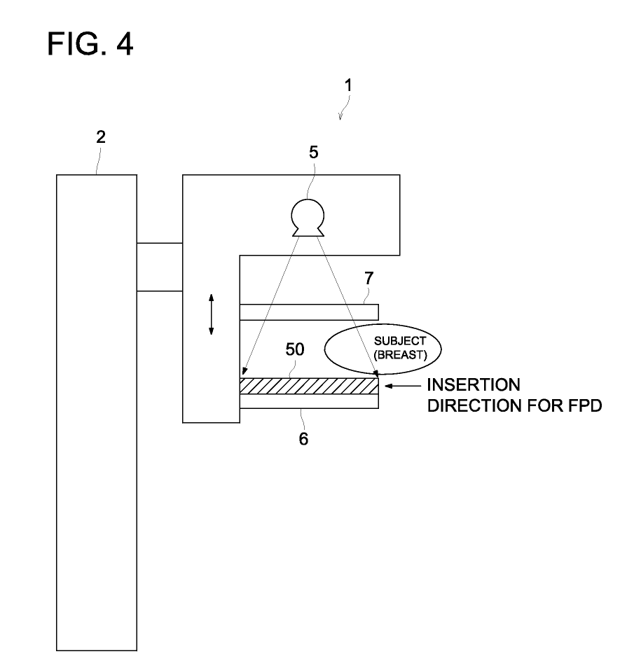

In the imaging section 1, X-ray source 5 that emits X-ray and imaging stand 6 on which a breast is to be placed are arranged to face each other, and pressure plate 7 that holds and presses a breast placed on the imaging stand 6 is provided. FIG. 4 shows a side view of the imaging section 1. As shown in FIG. 4, the imaging section 1 is constructed so that FPD 50 may be mounted on imaging stand 6, and a holder (not shown) for fixing the FPD 50 on the imaging stand 6 is formed.

When imaging is conducted in the imaging direction CC, the imaging is conducted under the state wherein the imaging section 1 is erected (rotation angle of 0°), while, when imaging is conducted in the imaging direction MLO, the imaging is conducted by rotating the imaging section 1 by about 30° from the state where the imaging section 1 is erected.

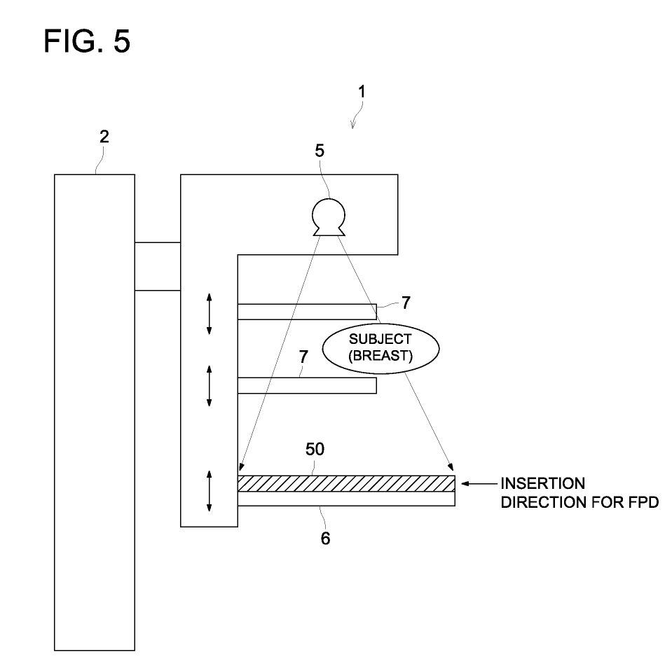

Further, when employing a phase contrast method as a method of imaging a breast image, the imaging section 1 has the structure shown in FIG. 5. The phase Contrast method is an imaging method to provide a distance between a patient and a detector (FPD) for enlargement, and a breast image having high resolution can be obtained by this method. Therefore, as shown in FIG. 5, two pressure plates 7 are provided in the imaging section 1, and imaging stand 6 is arranged below the pressure plates. In the case of the phase contrast method, a size of FPD 50 used for imaging is greater (in general, size (8 in.×10 in.) is used in the case of an ordinary imaging method, and size (14 in.×17 in.) is used in the case of the phase contrast method), imaging stand 6 is formed to have greater area.

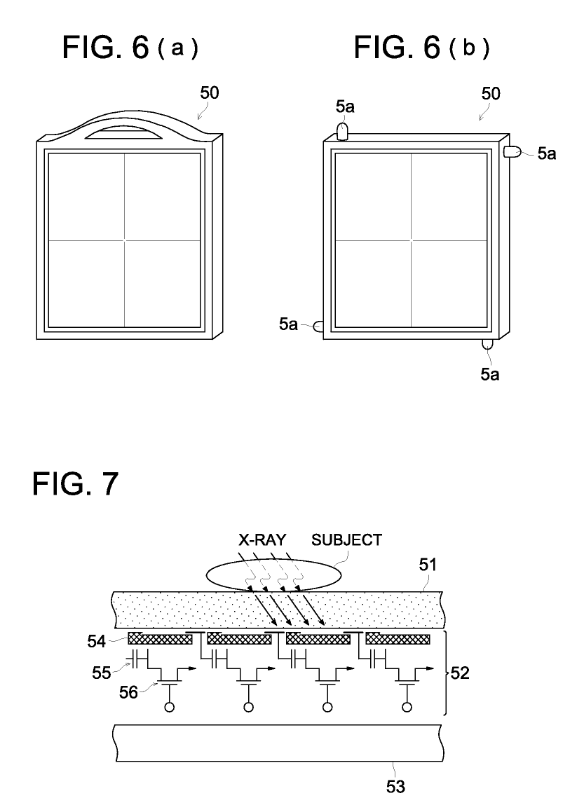

FPD 50 is an image generating device that generates data of breast images by generating electric signals corresponding to an irradiation dose of X-rays. Though the FPD 50 is formed to be in a rectangular shape, FPD having a handle portion on its one side as shown in FIG. 6 (a) is preferable for making the direction for mounting on imaging apparatus 10 (direction of arrangement) to be the same constantly. In addition, it is preferable to have the structure that makes the mounting direction to be constant, by giving a color or providing irregularity on one side that is held by a technician. Due to this, conditions for generating breast images in each imaging can be unified. Namely, the direction for reading breast image data from FPD is the same for each imaging, and a chest wall position comes to the same side on a rectangle-shaped image for reading.

Or, it is also possible to arrange so that switch 5a is provided on each of four sides of FPD 50, and a pressure that is applied on the switch 5a on one side that comes in contact with imaging apparatus 10 when FPD 50 is loaded on imaging apparatus 10 is detected, and information of the direction for loading is stored in FPD 50 accordingly, as shown in FIG. 6 (b). When the information of the direction for loading thus stored is used as correction information in the case of reading electric signals later, the conditions of generating breast images can be unified (a chest wall position can be made to be: on a prescribed side of rectangle-shaped image data). Specifically, there is conducted reading control such as controlling the direction to start reading of breast image data so that one side that is brought into contact with imaging apparatus 10 may be positioned constantly at the left side, for example, based on correction information. When the aforesaid structure is employed, the loading direction does not need to be controlled by a technician, resulting in excellent working efficiency.

Further, when using a mere rectangular FPD 50 having neither a handle nor a switch, it is also possible to employ the structure wherein a technician can operate to input into FPD 50 the information of the direction for loading FPD 50 in the course of imaging such as information telling which side among four sides is brought into contact with a patient. On FPD 50, the information of the direction for loading thus inputted is stored as correction information in the case of image reading, and reading control is conducted so that the condition for generating each breast image may be the same, in the same way as in the case of the structure by the switch stated above.

Next, internal constitution of FPD 50 will be explained as follows.

FPD 50 includes an indirect conversion type and a direct conversion type. The indirect conversion type is of a method wherein incident X-rays are converted into light once, and then, electric signals corresponding to an amount of that light are generated, while, the direct conversion type is of a method wherein incident X-rays are converted directly into electric signals without optical conversion. In the present embodiment, an example of FPD 50 employing the indirect conversion type, which also applies to the direct conversion type.

FIG. 7 shows a partial sectional view of FPD 50 of an indirect conversion type.

As shown in FIG. 7, FPD 50 is composed of phosphor layer 51, photoelectric conversion layer 52 and substrate 53. The phosphor layer 51 is formed in a way where phosphor material composed of phosphor and a binder is coated on a support, and it absorbs incident X-rays to emit fluorescence corresponding to intensity of the X-rays. The photoelectric conversion layer 52 is formed in a way where plural pixels each being one pixel unit composed of photoelectric conversion element 54, capacitor 55 and thin film transistor 56 are arranged on substrate 53 in a matrix form. The photoelectric conversion element 54 is one that generates electric charges corresponding to an amount of fluorescence in the phosphor layer 51, and the thin film transistor 56 is a switching element that switches reading of electric signals accumulated in capacitor 55.

X-rays having entered the phosphor layer 51 are converted into fluorescence, then, are converted into electric charges corresponding to its intensity of fluorescence (proportional to X-ray intensity) in the photoelectric conversion layer 52, and are accumulated in capacitor 55 as electric signals. Then, in the course of reading, the thin film transistor 56 is caused to be in the state of ON in accordance with instructions coming from an unillustrated control section, whereby, electric signals accumulated in capacitor 55 are converted into digital data through A/D converting section (not shown), and data of breast images are generated.

Next, control apparatus 30 will be explained as follows.

The control apparatus 30 conducts imaging control based on imaging order information, and controls breast images which have been read by FPD 50.

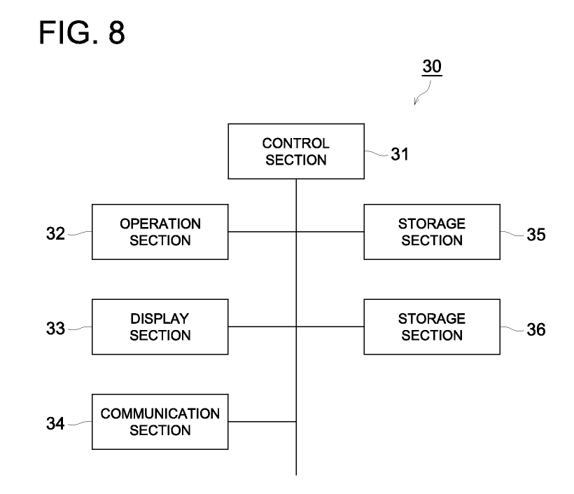

FIG. 8 shows an internal structure of control apparatus 30.

As shown in FIG. 8, the control apparatus 30 is composed of control section 31, operation section 32, display section 33, communication section 34, storage section 35 and image memory 36.

The control section 31 is composed of CPU (Central Processing Unit) and RAM (Random Access Memory), and it develops control programs stored in storage section 35 by CPU on RAM, and controls processing operations of each section on a panoptic basis, in accordance with the control program.

In the present embodiment, when a plurality of breast images are acquired per one patient, the control section 31 specifies a breast image of MLO among respective breast images and specifies a breast region in a breast image of MLO, based on instruction information inputted through operation section 32. Then, the control section 31 discriminates a breast region of other breast image based on the order of acquisition (order of imaging) of each breast image and on the breast image of MLO. Further, the direction of imaging for other breast image is discriminated based on imaging order information and on a breast image of MLO.

The operation section 32 is an operation device such as a key board, a mouse and a touch panel constructed solidly with display section 33, and it generates operation signals corresponding to the operation to output them to control section 31.

The display section 33 is composed of an indication display such as LCD (Liquid Crystal Display) and it displays display information such as results of processing and messages in addition to viewing surfaces of various types of operations such as a list screen of imaging order information and a viewing surface of results of imaging for breast images inputted from FPD 50.

The communication section 34 is composed of an interface for communication such as a network interface card and a modem, and it conducts transmitting and receiving for data with outer equipment such as imaging apparatus 10 and FPD 50. For example, it receives imaging order information issued, before imaging, in an unillustrated HIS (Hospital Information System) and RIS (Radiology Information System). Or, it receives data of breast images read out of FPD 50.

The storage section 35 stores data such as control program, parameter necessary for carrying out the control program, or results of processing.

The image memory 36 stores breast images received from FPD 50.

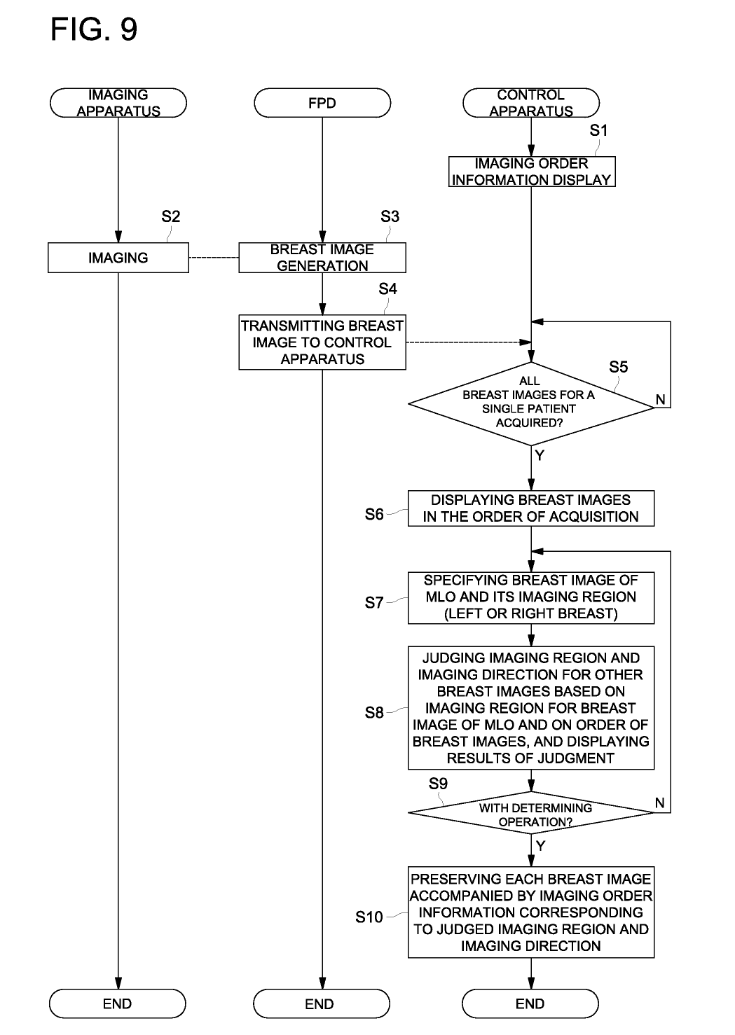

Next, operations of breast imaging system 100 in the present embodiment will be explained together with imaging operations of a technician, referring to FIG. 9.

In the breast imaging system 100, patients to be imaged are list-displayed on control apparatus 30 first, based on imaging order information, as shown in FIG. 9. After a patient to be imaged is selected from the list-displayed patients by a technician, the imaging order information corresponding to the selected patient is displayed (step S1).

Incidentally, in the present embodiment, the following explanation will be given under the assumption that the imaging order information instructs to image in MLO and CC directions for each of a left breast region and a right breast region for one patient.

A technician confirms a patient to be imaged, an imaging region and an imaging direction based on imaging order information displayed in control apparatus 30, and then, moves to an imaging room where imaging apparatus 10 is installed, while carrying one FPD 50 to be used for imaging. Then, the technician loads the cassettes c on imaging stand 60 of the imaging apparatus 10. In this case, the technician loads cassette c so that inserts FPD 50 while holding a handle portion of FPD 50 with a right hand with an imaging surface of FPD 50 facing upward, to load on imaging stand 6. Incidentally, if a direction for loading FPD 50 is made to be the same constantly, any direction can be used for loading.

Since the imaging apparatus 10 becomes ready for imaging, the technician causes a patient to stand to adjust an imaging region and an imaging direction of a patient to be imaged, and then, conducts instruction operations for imaging through an operation section of the imaging apparatus 10. On the imaging apparatus 10, X-ray irradiation corresponding to instruction for imaging is conducted, and imaging is conducted (step S2).

When imaging in one imaging direction for a breast region of any one of left and right is terminated in the aforesaid way, electric signals corresponding to an amount of X-rays transmitted through a patient are generated and accumulated on FPD 50. After that, processing of reading the accumulated electric signals is conducted, and breast image data are generated (step S3). These breast image data are caused to accompany, after information such as reading condition is written on a header of breast images thus read out, to be transmitted to control apparatus 10 (step S4). When processing to remove residual electric charges is terminated after completion of data reading, FPD 50 becomes ready to be used for succeeding imaging.

As stated above, processing that breast image data are transmitted to control apparatus 30 where the breast image data are generated is conducted four times in total for MLO direction and CC direction for left and right breasts. In this case, a technician images alternately for left and right breast regions for avoiding imaging continuously for a breast region on one side.

Then, if all breast image data instructed by imaging order information for one patient are acquired from FPD 50, in control apparatus 30 (step S5; Y), respective breast images acquired by control section 31 are arranged in the order of the acquisition to be displayed on display section 33 (step S6). In the present embodiment, the order of acquisition of the breast image data agrees with that of imaging, because of the arrangement where breast image data are generated and are transmitted to control apparatus 30 each time imaging is conducted.

Incidentally, it is also possible to constitute so that breast image data generated for each imaging are preserved successively in a memory built in PPD. In this case, after completion of all imaging operations for one patient, breast image data preserved in a memory (for example, breast image data for imaging of four times) are transmitted to control apparatus 30 in succession, beginning with breast image data taken in the first place. It is further possible to arrange to transmit to the control apparatus 30, beginning with data taken through imaging lastly.

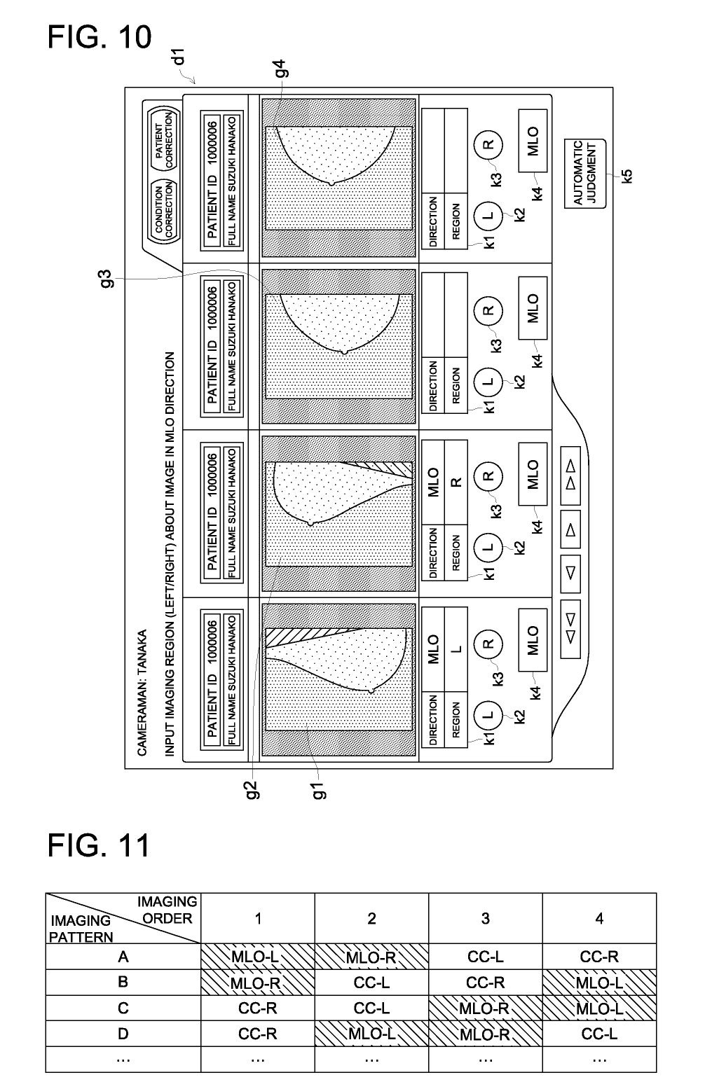

FIG. 10 shows an example of display by display section 33 equipped on control apparatus 30.

On image plane of imaging result d1 shown in FIG. 10, there are displayed four breast images g1-g4 which have been taken through imaging for the same patient, in a way to arrange them in the order of imaging from left to right on the image plane. Further, with respect to each breast image, there are provided display area k1 for an imaging direction and an imaged region, designating buttons k2 and k3 for imaged regions (left and right) and designating button k4 that designates MLO as an imaging direction.

A technician confirms image quality and others of each breast image on this image plane of imaging result d1, and determines a breast image of MLO on which an imaging direction and an imaged region can be discriminated, among the aforesaid breast images. Then, with respect to the breast image of MLO, the technician operates to press down designating button k4 of MLO, and further operates to press down left designating button k2 for a breast image of a left breast and to press down right designating button k3 for a breast image of a right breast. In this case, it is assumed that breast images of MLO are designated to be breast image g1 and breast image g2 and breast image g1 among them is designated to be a left breast and breast image g2 is designated to be a right breast.

On the control apparatus 30, pieces of information including imaging directions of MLO designated in accordance with operations of respective buttons k2-k4 and left and right breast regions are displayed on the display area k1 (step S7). The technician confirms designation information displayed on the display area k1, and operates to press down automatic discrimination button k5, if the designation is acceptable.

On the control apparatus 30, if the automatic discrimination button k5 is pressed down, imaging directions and breast regions of other breast images g3 and g4 are discriminated based on imaged regions of the designated breast images g1 and g2 of MLO and on the order of arrangement of respective breast images g1-g4, namely on the order of imaging. Incidentally, when discriminating only left and right, left or right of remaining three images can be discriminated automatically, by discriminating a left breast or a right breast of at least one of two MLO images.

How to discriminate will be explained specifically as follows.

In the example stated above, breast image g1 taken firstly in the order of imaging represents a left breast, and breast image g2 taken secondly in the order of imaging represents a right breast. Since imaging operations of breast regions are conducted alternately for left and right, it is discriminated so that an imaged region of breast image g3 taken thirdly in imaging order is a left breast, while, an imaged region of breast image g4 taken fourthly in imaging order is a right breast. Further, since the imaging directions designated by imaging order information in this occasion were MLO and CC, the imaging direction for other breast images g3 and g4 are discriminated to be CC.

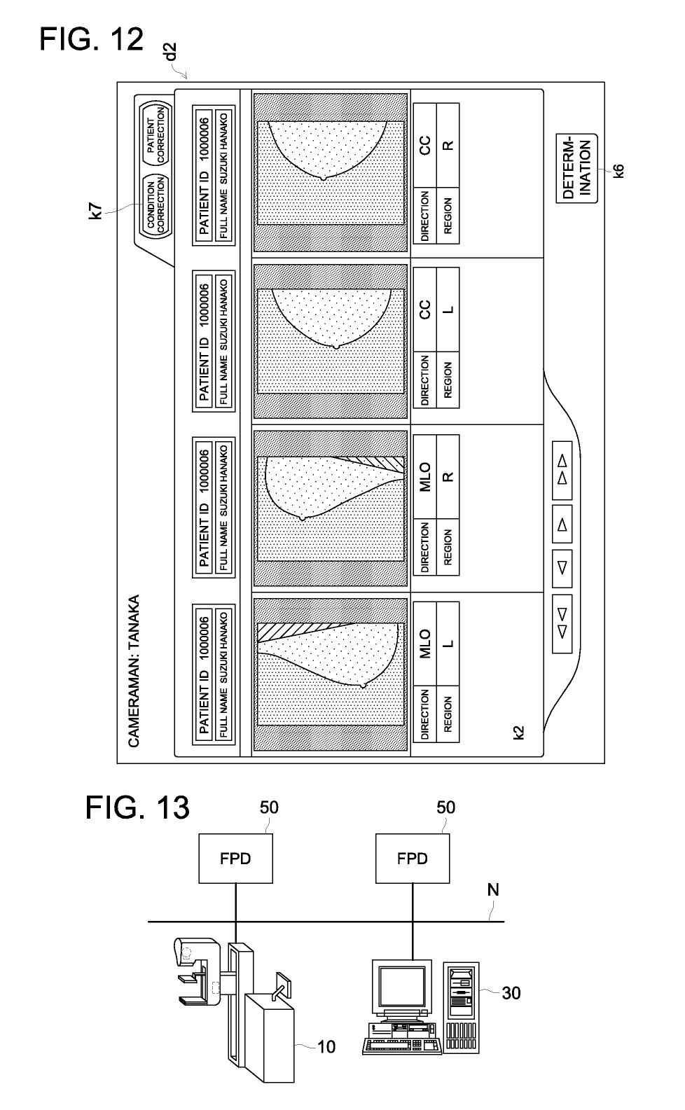

If it is possible to specify left or right for a breast, concerning a breast image of MLO, even in the case of an imaging pattern shown in FIG. 11, without being limited to the aforesaid imaging pattern, it is possible to discriminate left or right of other breast image to be alternate for left and right, based on the imaging order and on relationship between left and right of breast regions. Further, if there is only one direction in addition to MLO for imaging directions, another imaging direction is CC.

Results of discrimination for a breast region and an imaging direction about other breast images g3 and g4 are displayed in a way shown in FIG. 12 (step S8).

On image plane of discrimination result d2 shown in FIG. 12, information of breast regions and imaging direction of CC which are discriminated on display areas k1 and k2 for other breast images g3 and g4 are inputted automatically to be displayed. When the displayed contents are acceptable, a technician operates to press down enter button k6 that determines breast regions and imaging directions for respective breast images g1-g4. When the displayed breast regions and imaging direct ions need to be corrected, condition correction button k7 is pressed down.

On the control apparatus 30, when enter button k6 is pressed for determination (step S9; Y), imaging order information corresponding to imaged regions and imaging directions discriminated for respective breast images g1-g4 are caused to accompany as supplementary information of breast images g1-g4, and then, the breast images g1-g4 are preserved (step S10).

Incidentally, when condition correction button k7 is pressed down on image plane of discrimination result d2, a flow returns to step S7, and image plane of imaging result d1 shown in FIG. 10 is displayed again, and processes of step S7-step S9 are repeated.

In the present embodiment, by imaging alternately for left and right breast regions by changing imaging direction and thereby by generating data of breast images with FPD 50 each time imaging is conducted, to transmit them to control apparatus 30, or by transmitting to the control apparatus 30 successively beginning with old data (breast image data taken firstly) after completion of all operations of imaging, as stated above, it is possible to discriminate easily left or right of a breast region of other breast image, based on breast images of MLO. It is also possible to discriminate an imaging direction of other breast image based on information of breast images of MLO and imaging order. Owing to this, it is possible to cause corresponding imaging order information to accompany breast images without conducting registration of FPD 50, and to reduce a load of imaging operations for a technician substantially.

Further, since a breast region of the breast image of MLO is specified by designating operations of a technician, discrimination for left and right of breast regions based on judgment of the technician can be carried out.

Incidentally, the explanation given above is a suitable example to which the invention is applied, and the invention is not limited to the foregoing.

For example, although an example wherein FPD 50 and control apparatus 30 are connected on a basis of 1:1 has been explained, it is also possible to arrange so that they are connected on a network basis under the relationship of (communication connectable between any FPD and any control apparatus) with relationship between m (number of FPD devices) and n (number of control devices), as shown in FIG. 13. In this case, a technician can image while replacing FPD 50 alternately for each imaging, and FPD 50 conducts reading of breast images immediately after the imaging, and its time-lag is almost constant. Therefore, order for the control apparatus 30 to acquire breast images from each FPD 50 never disagree with imaging order.

Further, in the explanation above, although a breast image of MLO and its imaged region are discriminated by displaying a breast image and by causing a technician to designate the breast image of MLO and its imaged region, it is also possible to arrange to conduct image analyses for breast images on the control apparatus 30 side and thereby to conduct automatic discrimination, without being limited to the foregoing. Breast images with the same imaging direction and the same imaged region are to be similar (a chest wall is positioned on the prescribed side of a rectangle-shaped image), because all of the breast images to be taken through imaging are under the same imaging condition and the same reading condition. Therefore, it is possible to prepare a pattern image of a right breast of MLO and a pattern image of a left breast of MLO (image on which a pectoral muscle is positioned above or below the aforesaid prescribed side) in advance, and thereby to compare these pattern images with breast images taken through imaging for judgment.

Specifically, a pattern image of a left breast is first compared with a breast image taken through imaging. As a result of the comparison, if the pattern image of the left breast agrees with the breast image taken through imaging, the breast image is discriminated to be a left breast. On the other hand, if the pattern image of the left breast does not agree with the breast image taken through imaging, a flow moves to comparison with a pattern image of a right breast. If the pattern image of the right breast agrees with the breast image taken through imaging, the breast image is discriminated to be R. In this way, it is possible to discriminate automatically LR of the breast image taken through imaging. When this automatic discrimination is carried out, a technician does not need to operate designation for breast images of MLO, which reduces a load of operations.

Incidentally, when automatic discrimination is conducted, the same imaging condition (a prescribed side of FPD is positioned at a chest wall) for positioning a chest wall on a prescribed side of the rectangle-shaped image which has been read is not always necessary, and positional relationship between FPD and a chest wall is loosened (any of four sides can be made to face a chest wall). However, a capacity of pattern images to be stored is sometimes increased, and a period of time required for pattern recognition is sometimes increased.

In this case, when causing left and right breast images to agree with each other on the breast images of MLO, mirroring processing of breast images is needed, and therefore, rotating processing only for breast images cannot make both left and right to agree with each other. By utilizing this, LR of breast images are discriminated.

Namely, the pattern image of the left breast is Compared with a breast image taken through imaging. As a result of the comparison, if the pattern image of the left breast agrees with a breast image taken through imaging, the breast image is discriminated to be L. On the other hand, if they disagree with each other, a pattern image of the left breast is rotated successively by 90°, or a pattern image which is rotated successively by 90° is prepared in advance, and this pattern image is compared again with the breast image taken through imaging. This operation is repeated four times, and if they disagree with each other, a pattern image of the right breast is compared. Then, the breast image taken through imaging is compared with the pattern image of the right breast in the same way as in the case of the pattern image of the left breast stated above, whereby, a left breast or a right breast can be discriminated.

### Second Embodiment

Breast imaging system 100 in the Second Embodiment is of the structure wherein CR cassette (hereinafter, called a cassette simply) having therein a built-in phosphor plate is used in place of FPD S0 used in the First Embodiment. Incidentally, in the explanation of the Second Embodiment, the same symbols as those in the First Embodiment are given to the same constituent components, and illustrations for them are omitted.

Even in the present embodiment, under the assumption that imaging is conducted alternately for left and right, and the direction of arrangement of the cassette in the case of imaging and the direction of insertion in the case of reading are the same, if it is possible to specify the breast image of MLO that can discriminate the breast region whether it is left or right among plural breast images for one patient, it is possible to discriminate left or right of a breast region and an imaging direction for other breast images, based on information of imaging sequence and imaging order.

Based on the foregoing, there will be explained an example wherein imaging is conducted under the same condition (a prescribed side of a rectangle-shaped cassette is caused to face a chest wall of a patient), and reading of breast images under the same condition in the aforesaid sequence (a direction of insertion of a prescribed side of a cassette facing a chest wall of a patient is made to be constant), in the present embodiment.

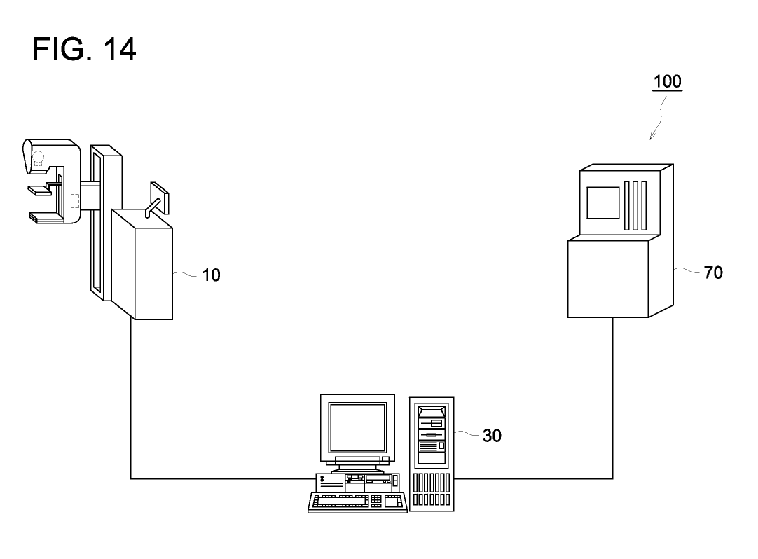

FIG. 14 shows the system structure of breast imaging system 100.

The breast imaging system 100 is composed of imaging apparatus 10, control apparatus 30 and reading apparatus 70, and the imaging apparatus 10 and the reading apparatus 70 are connected to the control apparatus 30.

The imaging apparatus 10 is one wherein a breast of a patient is subjected to X-ray imaging as a subject, and its X-ray images (breast images) are recorded on each cassette, respectively. Incidentally, the imaging apparatus 10 in the present embodiment is of the same structure as in the imaging apparatus (see FIG. 3) used in the first embodiment, excepting that a cassette holder (not shown) for fixing cassette c on imaging stand 6 is formed on imaging section 1, therefore, detailed explanation will be omitted here.

The cassette c is one having therein a built-in phosphor plate that absorbs X-rays transmitted through a patient and thereby records its X-ray image. Incidentally, the cassette c includes a cassette of an ordinary type that can be used also for regions to be imaged other than a breast and a cassette that is used exclusively for a breast. Any type among those stated above can be used, and for the purpose of unifying a direction of arrangement of cassette c on imaging stand 6, it is preferable to give a color, to provide irregularity or to change a material for one side of the cassette c so that a technician may discriminate that side to be positioned at the patient side. In the case of the cassette to be used exclusively for the breast, a phosphor plate is formed so that the aforesaid one side of the cassette may cover a chest wall of a patient up to the margin in the case of imaging, and it is general that the aforesaid actions are taken so that one side to be brought into contact with a patient may be discriminated.

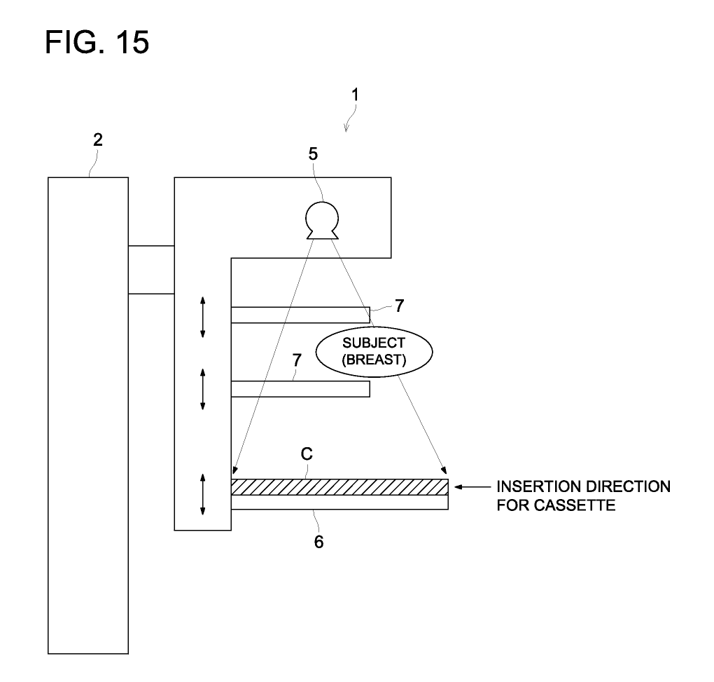

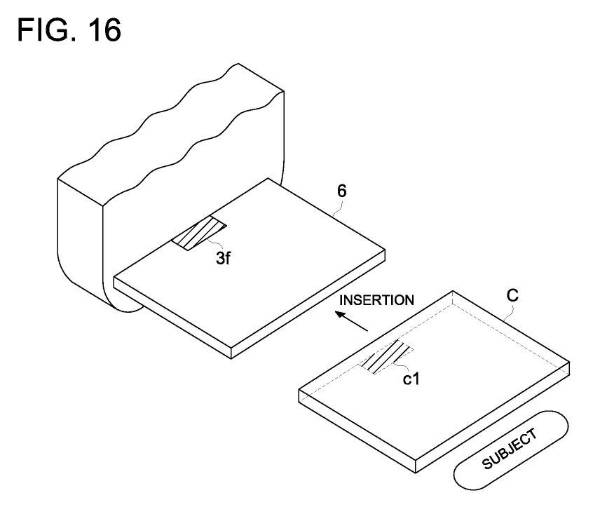

When employing a phase contrast method as an imaging method for breast images, imaging section 1 has a structure shown in FIG. 15. The phase contrast method is an imaging method for conducting enlargement by providing a distance between a patient and a detector (cassette), which makes it possible to obtain breast images with high resolution. Therefore, as shown in FIG. 15, two pressure plates 7 are provided on imaging section 1, and imaging stand 6 is arranged below the pressure plates. In the case of the phase contrast method, a size of cassette c to be used for imaging is large (in general, a size of (8×10 in.) is used for an ordinary imaging method, and a size of (14×17 in.) is used for a phase contrast method), and imaging stand 6 is formed to become greater in terms of an area.

Next, a bar-code to be provided on cassette c will be explained as follows.

On cassette c, there is generally provided peculiar ID as discrimination information, and this ID is converted into a bar-code to be displayed on the other side that is opposite to one side that comes in contact with a patient on cassette c, as shown in FIG. 6. Corresponding to this, ID reading section 3f that reads a bar-code is provided at the position corresponding to the portion (this is called ID section c1) where a bar-code is indicated when cassette c is arranged. When ID section c1 of cassette c placed on imaging stand 6 can be read correctly by this ID reading section 3f, the cassette c is judged to be loaded in the correct direction, while, when ID section c1 was not read, the direction of arrangement of the cassette c is judged to be wrong.

Next, control apparatus 30 will be explained as follows.

The control apparatus 30 controls imaging based on imaging order information, and controls breast images which have been read by reading apparatus 70.

The control apparatus 30 in the present embodiment is of the structure which is the same as that of the control apparatus in the first embodiment, and the control apparatus 30 is composed of control section 31, operation section 32, display section 33, communication section 34, storage section 35 and image memory 36, as shown in FIG. 8.

The control section 31 is composed of CPU, RAM and others, and it develops a control program stored in storage section 35 by CPU on RAM, and controls processing operations of respective sections collectively in accordance with the control program.

In the present embodiment, when plural breast images for one patient are acquired, the control section 31 specifies breast images of MLO among respective breast images and breast regions of breast images of MLO, based on designation information inputted through operation section 32. Then, it discriminates left and right of breast regions of other breast images based on acquisition order of respective breast regions and on at least one breast image of MLO.

The operation section 32 is an operation device such as a touch panel constructed integrally with a key board, a mouse and display section 33, and it generates operation signals corresponding to operations to output them to the control section 31.

The display section 33 is constructed to be equipped with an expression display such as LCD, and it displays expression information such as processing results and messages in addition to various types of operation image planes such as a list of image planes of imaging order information and imaging result image plane of breast images inputted from reading apparatus 70.

The communication section 34 is constructed to be equipped with an interface for communication such as a network interface card and a modem, and conducts transmitting and receiving of data with outer equipment such as imaging apparatus 10 and reading apparatus 70. For example, it receives imaging order information issued in unillustrated HIS and RIS before imaging. Or, it transmits to reading apparatus 70 the instruction information concerning image reading instructed in imaging order information, and receives from the reading apparatus 70 the data of breast images which were read in the reading apparatus 70.

The storage section 35 stores data such as parameters necessary for control program and for execution of the control program, or data of results of processing.

The image memory 36 stores breast images received from the reading apparatus 70.

Next, the reading apparatus 70 will be explained.

The reading apparatus 70 is an image generating device that conducts reading of breast images recorded on cassette c, and generates breast image data. The reading apparatus 70 is constructed to be equipped with a reading section that reads breast images recorded on cassette c by applying excitation light on a phosphor plate of loaded cassette c and by converting accelerated light photoelectrically, and generates breast images, a communication section to transmit the generated breast image data to control apparatus 30 and an operation section.

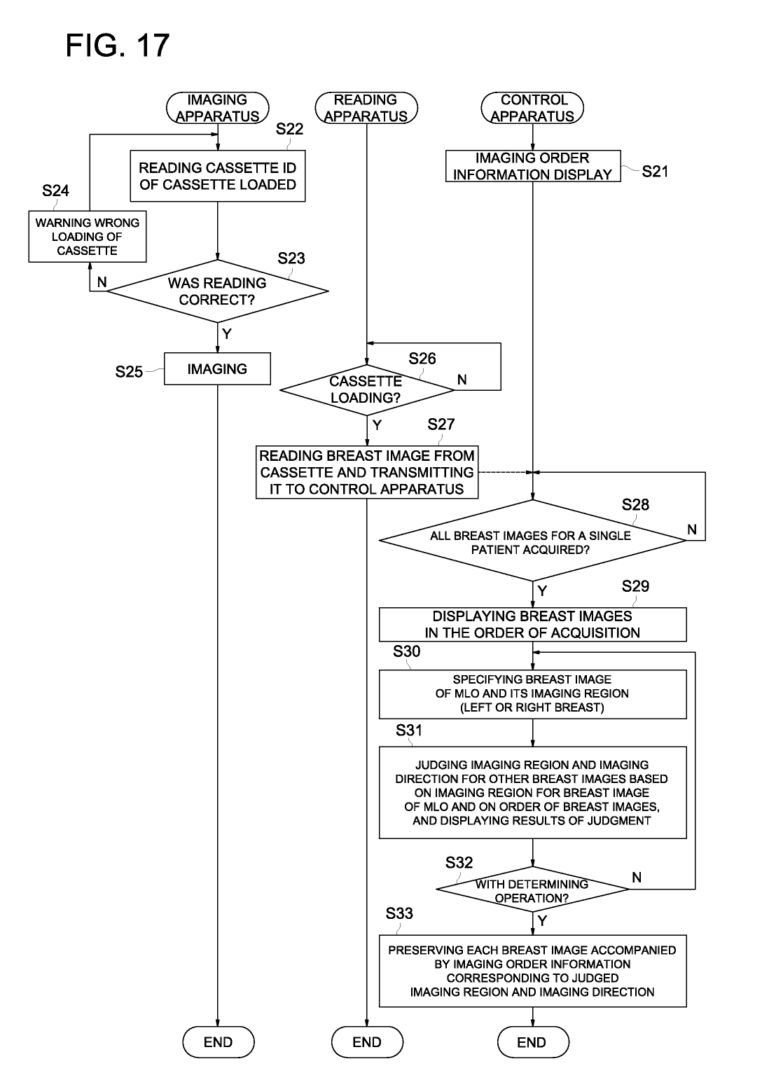

Next, operations of the aforesaid breast imaging system 100 will be explained together with imaging work of a technician, referring to FIG. 17.

In the breast imaging system 100, patients to be imaged are list-displayed on control apparatus 30 first, based on imaging order information, as shown in FIG. 17. After a patient to be imaged is selected from the list-displayed patients by a technician, the imaging order information corresponding to the selected patient is displayed (step S21).

Incidentally, in the present embodiment, the following explanation will be given under the assumption that the imaging order information instructs to image in MLO and CC directions for each of a left breast region and a right breast region for one patient.

A technician makes sure of a patient to be imaged, a region to be imaged and an imaging direction in accordance with imaging order information displayed on control apparatus 30, and moves to an imaging room where imaging apparatus 10 is installed, carrying cassettes c for the number of times of imaging. Then, the technician mounts cassette c on imaging stand 6 of imaging apparatus 10. In this case, the cassette c is mounted so that ID section c1 indicated on the cassette c may agree positionally with a reading position of ID reading section 3f on the imaging apparatus 10 side.

In the imaging apparatus 10, the ID reading section 3f conducts reading of ID section c1 of the mounted cassette c (Step S22), and when the reading is not correct (step S23; N), the cassette c is judged to be mounted wrongfully in terms of a direction, and a warning to the effect of wrong mounting is given (step S24). Any warning method such as displaying a warning message or conducting voice guide may be used. A technician mounts cassette c again after correcting the direction of mounting in accordance with the warning.

Then, when ID section c1 of the mounted cassette c is read correctly by the ID reading section 3f (Step S23; Y), the imaging apparatus 10 becomes ready for imaging. Therefore, a technician causes a patient to stand at an imaging position and operates to instruct imaging through an operation section of the imaging apparatus 10, after adjusting the imaging region to be imaged and the imaging direction. In the imaging apparatus 10, irradiation of X-rays is conducted in accordance with imaging instruction, and imaging is carried out (step S25).

After the imaging in one direction is completed for any one of a left breast and a right breast, in the aforesaid way, the technician mounts on reading apparatus 70 the cassette c on which breast images are recorded through imaging, and operates to instruct a start of reading. Incidentally, for the purpose of unifying conditions for reading breast images from cassette c, the direction for mounting cassette c is made to be the determined fixed direction constantly, by, for example, mounting cassette c so that its one side where ID section c1 of cassette c is provided may come to the forefront side, or by mounting cassette c so that its one side that is colored may be held by a technician. (ID reading section also functions as a judging section that judges whether the cassette c is mounted in this fixed direction or not.)

In the reading apparatus 70, when cassette c is mounted (step S26; Y), reading of breast images recorded on the mounted cassette c is started. Then, when reading is completed, information such as the condition for reading is written on a header of the generated breast images to be caused to accompany, and is transmitted to control apparatus 10 (step S27).

Imaging operations to conduct reading of cassette c immediately after imaging are conducted continuously for the total four times in MLO and CC directions for a left breast and a right breast, as stated above. In this case, a technician conducts imaging alternately for left and right breast regions, for avoiding that imaging is continuously conducted only for a breast region on one side (to reduce pains caused by a breast on one side that is pressed continuously).

Then, if all breast image data instructed for imaging by imaging order information for one patient are acquired from reading apparatus 70, in control apparatus 30 (step S28; Y), respective breast images acquired by control section 31 are arranged in the order of the acquisition to be displayed on display section 33 (step S29). In the present embodiment, the order of acquisition of the breast images agrees with that of imaging, because the number of reading apparatus 70 is only one, and reading of cassette c is conducted for each imaging.

In the present embodiment, a method of display of breast images on display section 33 is the same as that in the First Embodiment, and its example of display is shown in FIG. 10.

In image plane of imaging result d1 shown in FIG. 10, a technician confirms image quality of each breast image, and makes judgment for breast image of MLO capable of being discriminated about an imaging direction and an imaged region among the aforesaid breast images. With respect to breast images of MLO, the technician presses down designating button k4 of MLO, and further presses down left designating button k2 for breast images of a left breast, and presses down right designating button k3 for breast images of a right breast. In this case, it is assumed that breast images of MLO are designated to be breast image g1 and breast image g2, and among them, breast image g1 is designated to be a left breast, and breast image g2 is designated to be a right breast.

On the control apparatus 30, pieces of information of an imaging direction of MLO and of left and right breast regions designated in accordance of operations of respective buttons k2-k4 are displayed on display area k1 (step S30). A technician confirms designated information displayed on display area k1, and then, presses down automatic discriminating button k5 positioned at a corner on the right side of an image plane if the designation is acceptable.

On the control apparatus 30, if the automatic discriminating button k5 is pressed down, imaging directions and breast regions of other breast images g3 and g4 are discriminated based on imaged regions of the designated breast images g1 and g2 of MLO and on the order of arrangement of respective breast images g1-g4, namely, on the order of imaging.

A method of discrimination will be explained specifically.

With respect to imaging, breast regions are imaged alternately for the left and the right. In the example stated above, breast image g1 taken firstly through imaging represents a left breast, and breast image g2 taken secondly through imaging represents a right breast. Therefore, an imaged region of breast image g3 taken through imaging thirdly is discriminated to be a left breast, and an imaged region of breast image g4 taken through imaging fourthly is discriminated to be a right breast. Further, the imaging direction for other breast images g3 and g4 is discriminated to be CC, because imaging directions designated by imaging order information this time were MLO and CC.

If it is possible to specify whether the breast image of MLO is for a left breast or a right breast, despite the imaging pattern shown in FIG. 11 without being limited to the aforesaid imaging pattern, it is possible to discriminate left or right of other breast images in a way to be left and right alternately, from relationship between the order of imaging and left and right of breast regions. Further, if the imaging direction is only one in addition to MLO, another imaging direction should be CC.

Results of discrimination for breast regions and imaging directions of other breast images g3 and g4 are displayed as shown in FIG. 12 (step S31).

On image plane of discrimination result d2 shown in FIG. 12r pieces of information of discriminated breast regions and imaging direction of CC are inputted automatically to be displayed on display areas k1 and k2 of other breast images g3 and g4. If the displayed contents are acceptable, a technician presses down enter button k6 that determines a breast region and an imaging direction for respective breast images g3 and g4. When the displayed breast region and the imaging direction need to be corrected, condition correction button k7 is pressed down.

In the control apparatus 30, when determination operations are conducted by enter button k6 (step S32; Y), imaging order information corresponding to discriminated imaged regions and imaging directions for respective breast images g1-g4 is caused to accompany as accompanying information of breast images g1-g4, and then, breast images g1-g4 are preserved in image memory 36 (step S33).

Incidentally, when the condition correction button k7 is pressed down on the image plane of discrimination result d2, a flow returns to processing in step S30, and image plane of imaging result d1 shown in FIG. 10 is displayed again. Then, processes of steps S30-S32 are repeated.

In the present embodiment, when left and right breast regions are imaged alternately by changing an imaging direction, then, when the cassette c used for the imaging is read for each imaging, and when the direction of arrangement of the cassette c in that imaging and the direction of reading the cassette c are made to be the same, it is possible to discriminate easily whether the breast region of other breast image is left or right, based on the breast image of MLO. Further, it is possible to discriminate the direction of imaging for other breast image based on the breast image of MLO and on the imaging order information.

Owing to this, it is possible to cause the corresponding imaging order information to accompany the breast image, without conducting cassette registration, and to reduce a load of imaging operations for a technician substantially.

Incidentally, the explanation given above is a preferable example to which the invention is applied, and the present invention is not limited to the aforesaid explanation.

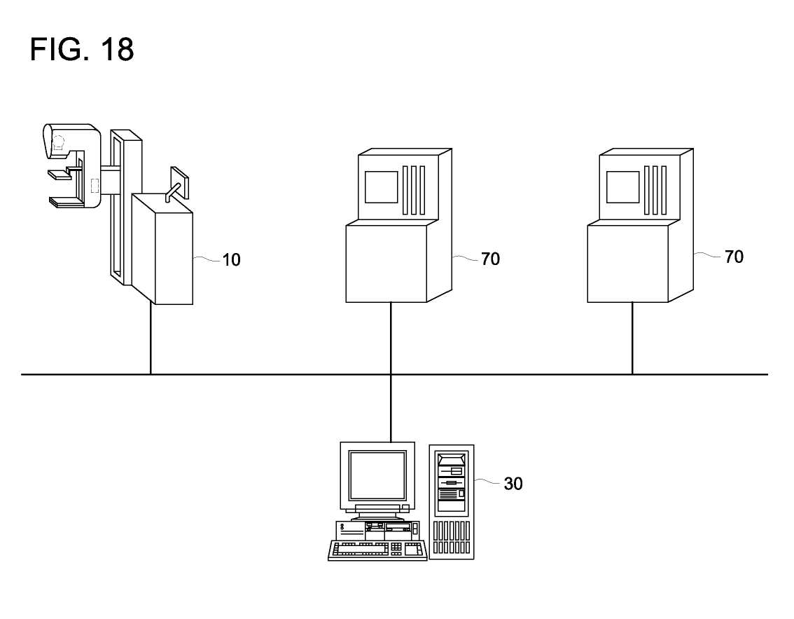

For example, an example wherein reading apparatus 70 and control apparatus 30 are connected to each other on 1:1 basis was explained in the explanation above, it is also possible to arrange to connect the reading apparatus to the control apparatus on a basis of m (number of reading apparatuses):n (number of control apparatuses) as shown in FIG. 18. In this case, a technician can mount cassette c which has been finished in terms of imaging on an optional reading apparatus 70, the order for the control apparatus 30 to acquire breast image data from each reading apparatus 70 does not disagree with the order of imaging, because cassette c is read and breast image data are generated for each imaging. Incidentally, there is considered an occasion wherein reading error is caused in reading apparatus 70 mounted firstly, and reading by another reading apparatus 70 mounted afterward is completed earlier. In this case, however, error information to the effect of reading error is transmitted from reading apparatus 70 on which reading error took place to the control apparatus 30 on which the order of arrangement of breast images acquired is changed based on the error information.

It is further possible to arrange so that the aforesaid discrimination of breast regions for left and right is carried out on a supplemental basis, after conducting cassette registration (causing imaging order information and cassette ID (bar-code ID) to correspond to each other). In this case, it is possible to discriminate correct breast region and imaging direction even when a wrong cassette is taken in the course of actual imaging, and thereby to correct to the contents corresponding to the actual results of imaging.

Further, in the explanation above, breast images are displayed, and breast images of MLO and imaged regions for them are designated by a technician, to discriminate a breast image of MLO and its imaged region. However, it is also possible to conduct image analyses for breast images on the control apparatus 30 side and thereby to carry out automatic discrimination, without being limited to the foregoing. Since all of the breast images to be taken are mounted on imaging apparatus 10 in the same direction and are loaded on reading apparatus 70 in the same direction, breast images in the same imaging direction and of the same imaged region should bear resemblance. Therefore, it is possible to discriminate them by preparing a pattern image of a right breast of MLO and a pattern image of a left breast of MLO in advance, and by comparing these pattern images with breast images taken through imaging.

Specifically, a pattern image of the left breast is compared with a breast image taken through imaging first. If the pattern image of the left breast agrees with the breast image taken through imaging as a result of the comparison, the breast image is discriminated to be left. On the other hand, if the pattern image of the left breast disagrees with the breast image taken through imaging, the comparison moves to one where a pattern image of the right breast is compared with a breast image taken through imaging. If the pattern image of the right breast agrees with the breast image taken through imaging, the breast image is discriminated to be R. By discriminating left or right of imaged breast automatically in this way, designating operations of a technician about breast images of MLO become unnecessary, which reduces a load of operations.

Incidentally, when automatic discrimination is conducted, the same imaging condition (a prescribed side of CR cassette is positioned at a chest wall) for positioning a chest wall on a prescribed side of the rectangle-shaped image which has been read is not always necessary, and positional relationship between CR cassette and a chest wall is loosened (any of four sides can be made to face a chest wall). However, a capacity of pattern images to be stored is sometimes increased, and a period of time required for pattern recognition is sometimes increased.

In this case, when causing left and right breast images to agree with each other on the breast images of MLO, mirroring processing of breast images is needed, and therefore, rotating processing only for breast images cannot make both left and right to agree with each other because a direction of CR cassette to be loaded on reading apparatus 70 can be controlled by the aforesaid reading function to be constant (for loading directions other than the fixed direction, a warning is announced under bar-code reading failure).

Namely, a pattern image of the left breast is compared with a breast image taken through imaging first. If the pattern image of the left breast agrees with the breast image taken through imaging as a result of the comparison, the breast image is discriminated to be L. On the other hand, if they disagree with each other, a pattern image of the left breast is rotated successively by 90°, or a pattern image which is rotated successively by 90° is prepared in advance, and this pattern image is compared again with the breast image taken through imaging. This operation is repeated four times, and if they disagree with each other, a pattern image of the right breast is compared. Then, the breast image taken through imaging is compared with the pattern image of the right breast in the same way as in the case of the pattern image of the left breast stated above, whereby, RL of breast images can be discriminated.

1. A method of discriminating left or right for breast regions, comprising:
generating data of plural breast images when a left breast region and a right breast region are alternately imaged in an order and are repeatedly imaged in various imaging directions in the same order by image generating sections arranged in a certain direction; and
specifying a breast image which is capable of being discriminated whether the imaged breast region is left or right among the plural breast images, and discriminating whether the left breast region or the right breast region is imaged concerning other breast images, based on the specified breast image and the order of the imaging of the plural breast images of a single patient.

2. The method of discriminating left or right for the breast region of claim 1,
wherein the breast image capable of being discriminated whether the imaged breast region is left or right is a breast image which is imaged in an oblique direction.

3. The method of discriminating left or right for the breast region of the claim 2,
wherein the plural breast images are those imaged in the oblique direction and in a vertical direction for each of the left breast region and the right breast region.

4. The method of discriminating left or right for the breast region of claim 2,
wherein, in the discriminating, an imaging direction for the other breast images are discriminated based on the breast image imaged in the oblique direction.

5. The method of discriminating left or right for the breast region of claim 4,
wherein, in the discriminating, when each breast image is aligned in the order of imaging, based on the breast region of the breast images imaged in the oblique direction, the breast regions of the other breast images are discriminated so that the breast regions of each breast image are aligned to alternate left and right.

6. The method of discriminating left or right for the breast region of claim 5,
wherein, in the discriminating, when a user inputs that the breast region of the breast image is left or right of the breast image concerning the breast image imaged in the oblique direction,
the breast regions of other breast images are discriminated whether they are left or right, based on information of left or right of the breast region inputted.

7. The method of discriminating left or right for the breast region of claim 5,
wherein, in the discriminating, after image analyses are conducted for each breast image, the breast image imaged in the oblique direction is discriminated based on the results of the image analyses, and the breast region of other breast images are discriminated left or right based on the breast image imaged in the oblique direction.

8. The method of discriminating left or right for the breast regions of claim 1, wherein the image generating sections comprise a cassette and a reading apparatus of the cassette.

9. The method of discriminating left or right for the breast regions of claim 1, wherein the image generating sections comprise a flat panel detector.

10. The method of discriminating left or right for the breast regions of claim 9, wherein the flat panel detector comprises an image arranging button.
